(19) United States
(12) Patent Application Publication
(10) Pub. No.: US 2004/0072743 A1
(43) Pub. Date: Apr. 15, 2004

(54) Pharmaceutical composition of modified pna molecules

(76) Inventors: Jeppe Viggo Christensen, Farum (DK); Edward Kristensen, Malov (DK)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/240,641

(22) PCT Filed: Apr. 6, 2001
(86) PCT No.: PCT/DK01/00238

(30) Foreign Application Priority Data
Apr. 6, 2000 (DK) PA 2000 00587

(51) Int. Cl. A61K 48/00

(52) U.S. Cl. 514/12

(57) ABSTRACT

The present invention concerns a pharmaceutical composition for use in combating infections.

The present invention concerns a pharmaceutical composition for use in combating infections. More particular the invention concerns pharmaceutical compositions comprising peptide nucleic acid (PNA) sequences, which are modified in order to obtain novel PNA molecules with enhanced anti-infective properties.

### BACKGROUND OF THE INVENTION

From the discovery of penicillin in the 1940's there has been an ever-growing search for new drugs. Many drugs or antibiotics have been discovered or developed from already existing drugs. However, over the years many strains of bacteria have become resistant to one or more of the currently available drugs, which were effective, drugs in the past. The number of antibiotic drugs currently being used by clinicians is more than 100.

Most antibiotics are products of natural microbic populations and resistant traits found in these populations can disseminate between species and appear to have been acquired by pathogens under selective pressure from antibiotics used in agriculture and medicine (Davies 1994). Antibiotic resistance may be generated in bacteria harbouring genes that encode enzymes that either chemically alter or degrade the antibiotics. Another possibility is that the bacteria encodes enzymes that makes the cell wall impervious to antibiotics or encode efflux pumps that eject antibiotics from the cells before they can exert their effects.

Because of the emergence of antibiotic resistant bacterial pathogens, there is an on-going need for new therapeutic strategies. One strategy to avoid problems caused by resistance genes is to develop anti-infective drugs from novel chemical classes for which specific resistance traits do not exist.

Antisense agents offer a novel strategy in combating diseases, as well as opportunities to employ new chemical classes in the drug design.

Oligonucleotides can interact with native DNA and RNA in several ways. One of these is duplex formation between an oligonucleotide and a single stranded nucleic acid. Another is triplex formation between an oligonucleotide and double stranded DNA to form a triplex structure.

Results from basic research have been encouraging, and antisense oligonucleotide drug formulations against viral and disease causing human genes are progressing through clinical trials. Efficient antisense inhibition of bacterial genes also could have wide applications; however, there have been few attempts to extend antisense technology to bacteria.

Peptide nucleic acids (PNA) are compounds that in certain respects are similar to oligonucleotides and their analogs and thus may mimic DNA and RNA. In PNA, the deoxyribose backbone of oligonucleotides has been replaced by a pseudo-peptide backbone (Nielsen et al. 1991) (FIG. 1). Each subunit, or monomer, has a naturally occurring or non-naturally occurring nucleobase attached to this backbone. One such backbone is constructed of repeating units of N-(2-aminoethyl)glycine linked through amide bonds. PNA hybridises with complementary nucleic acids through Watson and Crick base pairing and helix formation (Egholm et al. 1993). The Pseudo-peptide backbone provides superior hybridization properties (Egholm et al. 1993), resistance to enzymatic degradation (Demidov et al. 1994) and access to a variety of chemical modifications (Nielsen and Haaima 1997).

PNA binds both DNA and RNA to form PNA/DNA or PNA/RNA duplexes. The resulting PNA/DNA or PNA/RNA duplexes are bound with greater affinity than corresponding DNA/DNA or DNA/RNA duplexes as determined by Tm's. This high thermal stability might be attributed to the lack of charge repulsion due to the neutral backbone in PNA. In addition to increased affinity, PNA has also been shown to bind to DNA with increased specificity. When a PNA/DNA duplex mismatch is melted relative to the DNA/DNA duplex, there is seen an 8 to 20° C. drop in the Tm.

Furthermore, homopyrimidine PNA oligomers form extremely stable PNA₂-DNA triplexes with sequence complementary targets in DNA or RNA oligomers. Finally, PNA's may bind to double stranded DNA or RNA by helix invasion.

An advantage of PNA compared to oligonucleotides is that the PNA polyamide backbone (having appropriate nucleobases or other side chain groups attached thereto) is not recognised by either nucleases or proteases and are thus not cleaved. As a result, PNA's are resistant to degradation by enzymes unlike nucleic acids and peptides.

For antisense application, target bound PNA can cause steric hindrance of DNA and RNA polymerases, reverse transcription, telomerase and the ribosome's (Hanvey et al. 1992, Knudsen et a. 1996, Good and Nielsen 1998), etc.

A general difficulty when using antisense agents is cell uptake. A variety of strategies to improve uptake can be envisioned and there are reports of improved uptake into eukaryotic cells using lipids (Lewis et al. 1996), encapsulation (Meyer et al. 1998) and carrier strategies (Nyce and Metzger 1997, Pooga et al, 1998).

WO 99/05302 discloses a PNA conjugate consisting of PNA and the transporter peptide transportan, which peptide may be used for transport cross a lipid membrane and for delivery of the PNA into interactive contact with intracellular polynucleotides. U.S. Pat. No. 5,777,078 discloses a pore-forming compound which comprises a delivery agent recognising the target cell and being linked to a pore-forming agent, such as a bacterial exotoxin. The compound is administered together with a drug such as PNA.

As an antisense agent for microorganisms, PNA may have unique advantages. It has been demonstrated that PNA based antisense agents for bacterial application can control cell growth and growth phenotypes when targeted to Escherichia coli rRNA and mRNA (Good and Nielsen 1998a,b and WO 99/13893).

However, none of these disclosures discuss ways of transporting the PNA across the bacterial cell wall and membrane.

Furthermore, for bacterial application, poor uptake is expected, because bacteria have stringent barriers against foreign molecules and antisense oligomer containing nucleobases appear to be too large for efficient uptake. The results obtained by Good and Nielsen (1998a,b) indicate that PNA oligomers enter bacterial cells poorly by passive diffusion across the lipid bilayers.

U.S. Pat. No. 5,834,430 discloses the use of potentiating agents, such as short cationic peptides in the potentiation of antibiotics. The agent and the antibiotic are co-administered.

WO 96/11205 discloses PNA conjugates, wherein a conjugated moiety may be placed on terminal or non terminal parts of the backbone of PNA in order to functionalise the PNA. The conjugated moieties may be reporter enzymes or molecules, steroids, carbohydrate, terpenes, peptides, proteins, etc. It is suggested that the conjugates among other properties may possess improved transfer properties for crossing cellular membranes. However, WO 96/11205 does not disclose conjugates, which may cross bacterial membranes.

WO 98/52614 discloses a method of enhancing transport over biological membranes. e.g. a bacterial cell wall. According to this publication, biological active agents such as PNA may be conjugated to a transporter polymer in order to enhance the transmembrane transport. The transporter polymer consists of 6-25 subunits; at least 50% of which contain a guanidino or amidino sidechain moiety and wherein at least 6 contiguous subunits contain guanidino and/or amidino sidechains. A preferred transporter polymer is a polypeptide containing 9 arginine.

Thus, despite the promising results in the use of the PNA technology obtained previously, there is a great need of developing new PNA antisense drugs, which are effective in combating microorganisms.

### SUMMARY OF THE INVENTION

The present invention concerns a new strategy for combating bacteria. It has previously been shown that antisense PNA can inhibit growth of bacteria. However, due to a slow diffusion of the PNA over the bacterial cell wall a practical application of the PNA as an antibiotic has not been possible previously. According to the present invention, a practical application in tolerable concentration may be achieved by modifying the PNA by linking a peptide or peptide-like sequence, which enhances the activity of the PNA.

Surprisingly, it has been found out that by incorporating a peptide, an enhanced anti-infective effect can be observed. The important feature of the modified PNA molecules seems to be a pattern comprising in particular positively charged and lipophilic amino acids or amino acid analogues. An anti-infective effect is found with different orientation of the peptide in relation to the PNA-sequence.

Thus, the present invention concerns a modified PNA molecule of formula (I):

Pptide-L-PNA (I)

wherein L is a linker or a bond;

Peptide is any amino acid sequence and

PNA is a Peptide Nucleic Acid as defined above.

More particularly, the present invention concerns a modified PNA molecule of formula (I)

Peptide-L-PNA (I)

wherein Peptide is a cationic peptide or cationic peptide analogue or a functionally similar moiety, the peptide or peptide analogue having the formula (II):

C-(B-A)ₙ-D, (II)

Wherein A consists of from 1 to 8 non-charged amino acids and/or amino acid analogs;

B consists of from 1 to 3 positively charged amino acids and/or amino acid analogs;

C consists of from 0 to 4 non-charged amino acids and/or amino acid analogs;

D consists of from 0 to 3 positively charged amino acids and/or amino acid analogs;

n is 1-10; and

the total number of amino acids and/or amino acid analogs is from 3 to 20.

In one embodiment, The Peptide of the present invention contains from 2 to 60 amino acids. The amino acids can be negatively, non-charged or positively charged naturally occurring, rearranged or modified amino acids.

In a preferred embodiment of the invention the peptide contains from 2 to 18 amino acids, most preferred from 5 to 15 amino acids.

In another preferred embodiment of the invention A in formula (II) consists of from 1 to 6 non-charged amino acids and/or amino acid analogs and B consists of 1 or 2 positively charged amino acids and/or amino acid analogs. In another embodiment, A consists of from 1 to 4 non-charged amino acids and/or amino acid analogs and B consists of 1 or 2 positively charged amino acids and/or amino acid analogs.

In another aspect of the invention the modified PNA molecules are used in the manufacture of medicaments for the treatment or prevention of diseases such as bacterial and viral infections, cardiac or vascular diseases, metabolic diseases or immunological disorders or for disinfecting non-living objects.

In a further aspect, the invention concerns a composition for treating or preventing of diseases such as bacterial and viral infections, cardiac or vascular diseases, metabolic diseases or immunological disorders or for disinfecting non-living objects.

In yet another aspect, the invention concerns the treatment or prevention of diseases such as bacterial and viral infections, cardiac or vascular diseases, metabolic diseases or immunological disorders or for disinfecting of non-living objects.

In yet a further aspect, the present invention concerns a method of identifying specific advantageous antisense PNA sequences that may be used in the modified PNA molecule according to the invention.

In yet a further aspect, the present invention relates to a pharmaceutical composition comprising a molecule of formula (III):

PEP-L-A (III)

wherein

PEP is any amino acid sequence;

L is a linker or a bond and

A is an antisense agent.

The antisense agent is selected from the group consisting of PNA (Peptide Nucleic Acid) or a modified oligonucleotide being Locked Nucleoside Analogues (LNA) or morpholino analogues.

Locked Nucleoside Analogues (LNA) are described in International PCT Publication WO99114226 or in International PCT Publication WO98/03533.

Morpholino analogues are described in International PCT Publication WO98132467 all three PCT Publications are incorporated by reference.

### BRIEF DESCRIPTION OF THE DRAWINGS

FIG. 1 shows the chemical structure of DNA and PNA oligomers.

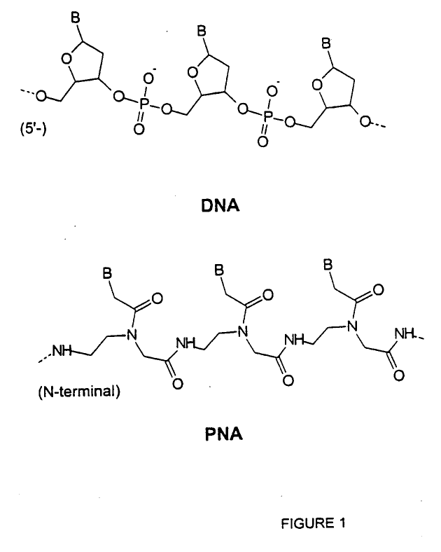

FIG. 2 shows the chemical structures of the different succinimidyl based linking groups used in the conjugation of the Peptide and PNA

FIG. 3 shows the principle in conjugation using SMCC

### DETAILED DESCRIPTION OF THE INVENTION

Antisense PNA's can inhibit bacterial gene expression with gene and sequence specificity (Good and Nielsen 1998a,b and WO 99/13893). The approach may prove practical as a tool for functional genomics and as a source for novel antimicrobial drugs. However, improvements on standard PNA are required to increase antisense potencies. The major limit to activity appears to be cellular entry. Bacteria effectively exclude the entry of large molecular weight foreign compounds, and previous results for in vitro and cellular assays seem to show that the cell barrier restricts antisense effects. Accordingly, the present invention concerns strategies to improve the activity of antisense potencies.

Without being bound by theory, it is believed that the short cationic peptides lead to an improved PNA uptake over the bacterial cell wall. It is believed that the short peptides act by penetrating the cell wall, allowing the modified PNA molecule to cross the cell wall to get access to structures inside the cell, such as the genome, mRNA's, the ribosome, etc. However, an improved accessibility to the nucleic acid target or an improved binding of the PNA may also add to the overall effect observed.

According to the invention, PNA molecules modified with short activity enhancing peptides enable specific and efficient inhibition of bacterial genes with nanomolar concentrations. Antisense potencies in this concentration are consistent with practical applications of the technology. It is believed that the present invention for the first time demonstrates that peptides with a certain pattern of cationic and lipophilic amino acids can be used as carriers to deliver agents and other compounds into micro-organisms, such as bacteria. Further, the present invention has made it possible to administer PNA in an efficient concentration, which is also acceptable to the patient.

Accordingly, the present invention concerns novel modified PNA molecules having the formula:

Peptide-L-PNA, wherein

L is a linker or a bond;

PNA is a peptide nucleic acid sequence; and

Peptide is a cationic peptide or peptide analogue or a functionally similar moiety, the peptide or peptide analogue having the formula:

C-(B-A)ₙ-D, wherein

A consists of from 1 to 8 non-charged amino acids and/or amino acid analogs;

B consists of from 1 to 3 positively charged amino acids and/or amino acid analogs;

C consists of from 0 to 4 non-charged amino acids and/or amino acid analogs;

D consists of from 0 to 3 positively charged amino acids and/or amino acid analogs;

n is 1-10; and

the total number of amino acids and/or amino acid analogs is from 3 to 20.

A preferred group of modified Peptide Nucleic Acids (PNA) molecule is the group wherein A consists of from 1 to 6 non-charged amino acids and/or amino acid analogs and B consists of 1 or 2 positively charged amino acids and/or amino acid analogs. In another preferred group A consists of from 1 to 4 non-charged amino acids and/or amino acid analogs and B consists of 1 or 2 positively charged amino acids and/or amino acid analogs.

By the terms “cationic amino acids and amino acid analogues” and “positively charged amino acids and amino acid analogues” are to be understood any natural or non-natural occurring amino acid or amino acid analogue which have a positive charge at physiological pH. Similarly the term “non-charged amino acids or amino acid analogs” is to be understood any natural or non-natural occurring amino acids or amino acid analogs which have no charge at physiological pH.

Among the positively charged amino acids and amino acid analogs may be mentioned lysine (Lys, K), arginine (Arg, R), diamino butyric acid (DAB) and ornithine (Om). The skilled person will be aware of further positively charged amino acids and amino acid analogs.

Among the non-charged amino acids and amino acid analogs may be mentioned the natural occurring amino acids alanine (Ala, A), valine (Val, V), leucine (Leu, L), isoleucine (Ile, I), proline (Pro, P), phenylanaline (Phe, F), tryptophan (Trp, W), methionine (Met, M), glycine (Gly, G), serine (Ser, S), threonine (Thr, T), cysteine (Cys, C), tyrosine (Tyr, Y), asparagine (Asn, N) and glutamine (Gln, Q), the non-natural occurring amino acids 2-aminobutyric acid, β-cyclohexylalanine, 4-chlorophenylalanine, norleucine and phenylglycine. The skilled person will be aware of further non-charged amino acids and amino acid analogs.

Preferably, the non-charged amino acids and amino acid analogs are selected from the natural occurring non-polar amino acids Ala, Val, Leu, Ile, Phe, Trp and Met or the non-natural occurring non-polar amino acids β-cyclohexylalanine, 4-chlorophenylalanine and norleucine.

The term “functionally similar moiety” is intended to cover all peptide-like molecules which functionally mimic the Peptide as defined above and thus impart to the PNA molecule the same advantageous properties as the peptides comprising natural and non-natural amino acids as defined above.

Examples of preferred modified PNA molecules according to the invention are (Lys Phe Phe)₃ Lys-L-PNA and any subunits thereof comprising at least three amino acids. One preferred Peptide is (Lys Phe Phe)₃ (SEQ ID NO: 8). Others include (Lys Phe Phe)₂Lys Phe (SEQ ID NO: 9), (Lys Phe Phe)₂Lys (SEQ ID NO: 10), (Lys Phe Phe)₂ (SEQ ID NO: 11), Lys Phe Phe Lys Phe (SEQ ID NO: 12), Lys Phe Phe Lys (SEQ ID NO: 13) and Lys Phe Phe.

Other preferred Peptides are FFRFFRFFR (SEQ ID NO: 14), LLKLLKLLK (SEQ ID NO: 15), LLRLLRLLR (SEQ ID NO: 16), LLKKLAKAL (SEQ ID NO: 17), KRRWPWWPWKK (SEQ ID NO: 18), KFKVKFWKK (SEQ ID NO: 19), LLKLLLKLLLK (SEQ ID NO: 20), LLKKLAKALK (SEQ ID NO: 21), and any subunits thereof comprising at least 3 amino acids whereof at least one amino acid is a positively charged amino acid.

A third group of preferred Peptides is RRLFPWWWPFRRVC (SEQ ID NO: 22), GRRWPWWPWKWPLIC (SEQ ID NO: 23), LVKKVATTLKKIFSKWKC (SEQ ID NO: 24), KKFKVKFVVKKC (SEQ ID NO: 25) and any subunit thereof comprising at least 3 amino acids whereof at least one amino acid is a positively charged amino acid.

A fourth group of preferred Peptides is magainis (Zasloff, M., Proc. Natl. Acad. Sci. USA, 84, p. 5449-5453 (1987)), for instance the synthetic magainin derivative GIGKFLHAAKKFAKAFVAEIMNS-NH₂ (SEQ ID NO: 26) as well as βamino-acid oligomers (β-peptides) as described by Porter, E. A. et al, Nature, 404, p. 565, (2000).

The number of amino acids in the peptide may be chosen between 3 and 20. It appears that at least 3 amino acids; whereof at least one is a positively charged amino acid is necessary to obtain the advantageous effect. On the other hand, the upper limit only seems to be limited by an upper limit of the overall size of the PNA molecule for the purpose of the practical use of said molecule. Preferably, the total number of amino acids is 15 or less, more preferable 12 or less and most preferable 10 or less.

The PNA molecule is connected to the Peptide moiety through a direct binding or through a linker. A variety of linking groups can be used to connect the PNA with the Peptide.

Linking groups are described in WO 96/11205 and WO98/52614, the content of which are hereby incorporated by reference.

Some linking groups may be advantageous in connection with specific combinations of PNA and Peptide.

Preferred linking groups are linkers selected from the group of pfphe (4-fluoro Phenylalanine), pnPhe (4-nitro Phenylalanine), chg (cyclohexyl Glycine), aha (6-amino-hexanoic acid), Gly (Glycine), b.ala (β-alanine), achc (Cis-4-aminocyclohexanoic acid), cha (β-cyclohexyl alanine), PheGly (Phenylglycine), g.abu (4-aminobutanoic acid), b.cypr (β-cyclopropyl alanine), m.achc (Cis-amino-cyclohexaneacetic acid), 5fPhe (Pentafluoro-Phenylalanine), pmba (4-aminomethyl-benzoic acid), ado ([2-(N-2-amino ethoxy)ethoxy] acetic acid), Nle (Norleucine), Nva (Norvaline), smcc (succinimidyl 4-(N-maleimidomethyl)cyclohexane-1-carboxylate), 4-aminobutyric acid, 4-aminocyclohexylcarboxylic acid, lcsmcc (succinimidyl 4-(N-maleimidomethyl)cyclohexane-1-carboxy-(6-amido-proate), mbs (succinimidyl m-maleimido-benzoylate), emcs (succinimidyl N-ε-maleimido-caproylate), smph (succinimidyl 6-(β-maleimido-propionamido) hexanoate, amas (succinimidyl N-(α-maleimido acetate), smpb (succinimidyl 4-(p-maleimidophenyl)butyrate) and adc (amino dodecanoic acid).

Any of these groups may be used as a single linking group or together with more groups in creating a suitable linker. Further, the different linking groups may be combined in any order and number in order to obtain different functionalities in the linker arm.

In a preferred embodiment the linking group is a combination of two or three of the above mentioned linkers.

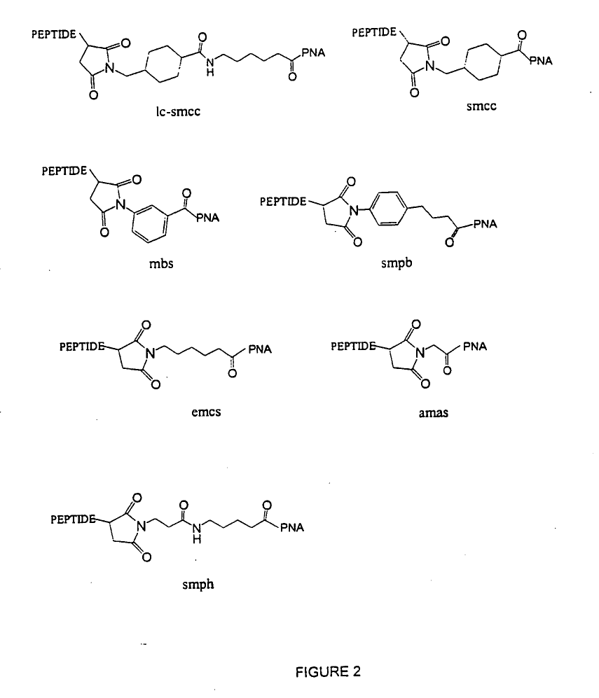

The chemical structures of the different succinimidyl based linking groups used in the conjugation of the Peptide and PNA is shown in FIG. 2.

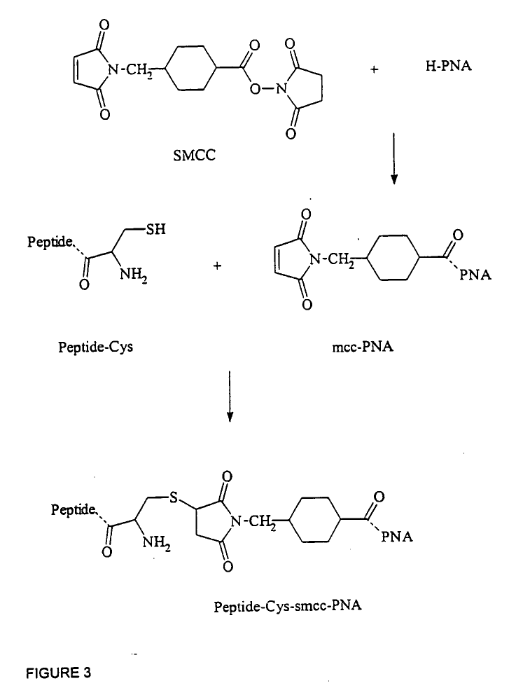

In the case SMCC (succinimidyl 4-(N-maleimidomethyl)cyclohexane-1-carboxylate) is used in the process of linking PNA to the peptide, it is necessary to add a cysteine (C) or a similar thiol containing moiety to the terminal end of the peptide (see FIG. 3). Additionally, amino acids, such as glycine, may be a part of the linker.

The Peptide is normally linked to the PNA sequence via the amino or carboxy end. However, the PNA sequence may also be linked to an internal part of the peptide.

The modified PNA molecule according to the present invention comprises a PNA oligomer of a sequence, which is complementary to at least one target nucleotide sequence in a micro-organism, such as a bacterium. The target may be a nucleotide sequence of any RNA, which is essential for the growth, and/or reproduction of the bacteria. Alternatively, the target may be a gene encoding a factor responsible for resistance to antibiotics. In a preferred embodiment, the functioning of the target nucleotide sequence is essential for the survival of the bacteria and the functioning of the target nucleic acid is blocked by the PNA sequence, in an antisense manner.

The binding of a PNA strand to a DNA or RNA strand can occur in one of two orientations, anti-parallel or parallel. As used in the present invention, the term complementary as applied to PNA does not in itself specify the orientation parallel or anti-parallel. It is significant that the most stable orientation of PNA/DNA and PNA/RNA is anti-parallel. In a preferred embodiment, PNA targeted to single strand RNA is complementary in an ant-parallel orientation.

In a another preferred embodiment of the invention a bis-PNA consisting of two PNA oligomers covalently linked to each other is targeted to a homopurine sequence (consisting of only adenine and/or guanine nucleotides) in RNA (or DNA), with which it can form a PNA₂-RNA (PNA₂-DNA) triple helix.

In another preferred embodiment of the invention, the PNA contains from 5 to 20 nucleobases, in particular from 7-15 nucleobases, and most particular from 8 to 12 nucleobases.

Peptide Nucleic Acids are described in WO 92/20702 and WO 92/20703, the content of which is hereby incorporated by reference.

Potential target genes may be chosen based on the knowledge about bacterial physiology. A target gene may be found among those involved in one of the four major process complexes: cell division, cell wall synthesis, protein synthesis (translation) and nucleic acid synthesis. A target gene may also be involved in antibiotic resistance.

A further consideration is that some physiological processes are primarily active in dividing cells whereas others are running under non-dividing circumstances as well.

Known target proteins in cell wall biosynthesis are penicillin binding proteins, PBPs, the targets of, e.g., the beta-lactam antibiotic penicillin. They are involved in the final stages of cross-linking of the murein sacculus.

E. coli has 12 PBPs, the high molecular weight PBPs: PBP1a, PBP1 b, PBP1c, PBP2 and PBP3, and seven low molecular weight PBPs, PBP 4-7, DacD, AmpC and AmpH. Only the high molecular weight PBPs are known to be essential for growth and have therefore been chosen as targets for PNA antisense.

Protein biosynthesis is an important process throughout the bacterial cell cycle. Therefore, the effect of targeting areas in the field of protein biosynthesis is not dependent on cell division.

Both DNA and RNA synthesis are target fields for antibiotics. A known target protein in DNA synthesis is gyrase. Gyrase acts in replication, transcription, repair and restriction. The enzyme consists of two subunits, both of which are candidate targets for PNA.

Examples of potential targets primarily activated in dividing cells are rpoD, gyrA, gyrB, (transcription), mrcA (ponA), mrcB (ponB, pbpF), mrdA, ftsl (pbpB) (Cell wall biosynthesis), ftsQ, ftsA and ftsZ (cell division).

Examples of potential targets also activated in non-dividing cells are infA, infB, infC, tufA/tufB, tsf, fusA, prfA, prfB, and prfC, (Translation).

Other potential target genes are antibiotic resistance-genes. The skilled person would readily know from which genes to choose. Two examples are genes coding for beta-lactamases in-activating beta-lactam antibiotics, and genes encoding chloramphenicol acetyl transferase.

PNA's against such resistance genes could be used against resistant bacteria.

Infectious diseases are caused by micro-organisms belonging to a very wide range of bacteria, viruses, protozoa, worms and arthropods and from a theoretical point of view PNA can be modified and used against all kinds of RNA in such micro-organisms, sensitive or resistant to antibiotics.

Examples of micro-organisms which may be treated in accordance with the present invention are Gram-positive organisms such as Streptococcus, Staphylococcus, Peptococcus, Bacillus, Listeria, Clostridium, Propionebacteria, Gram-negative bacteria such as Bacteroides, Fusobacterium, Escherichia, Klebsiella, Salmonella, Shigella, Proteus, Pseudomonas, Vibrio, Legionella, Haemophilus, Bordetella, Brucella, Campylobacter, Neisseria, Branhamella, and organisms which stain poorly or not at all with Gram's stain such as Mycobacteria, Treponema, Leptospira, Borrelia, Mycoplasma, Clamydia, Rickettsia and Coxiella,

The ability of PNA's to inhibit bacterial growth may be measured in many ways, which should be clear to the skilled person. For the purpose of exemplifying the present invention, the bacterial growth is measured by the use of a microdilution broth method according to NCCLS guidelines. The present invention is not limited to this way of detecting inhibition of bacterial growth.

To illustrate one example of measuring growth and growth inhibition the following procedure may be used:

Bacterial strain: E. coli K12 MG1655

Media: 10% Mueller-Hinton broth, diluted with sterile water.

10% LB broth diluted with sterile water.

100% Mueller-Hinton broth.

Trays: 96 well trays, Costar # 3474, Biotech Line AS, Copenhagen. (Extra low sorbent trays are used in order to prevent/minimize adhesion of PNA to tray surface).

A logphase culture of E. coli is diluted with fresh preheated medium and adjusted to defined OD (here: Optical Density at 600 nm) in order to give a final concentration of 5×10⁵ and 5×10⁴ bacteria/ml medium in each well, containing 200 μl of bacterial culture. PNA is added to the bacterial culture in the wells in order to give final concentrations ranging from 300 nM to 1000 nM. Trays are incubated at 37° C. by shaking in a robot analyzer, PowerWaveₓ, software KC^{4,} Kebo.Lab, Copenhagen, for 16 h and optical densities are measured at 600 nM during the incubation time in order to record growth curves. Wells containing bacterial culture without PNA are used as controls to ensure correct inoculum size and bacterial growth during the incubation. Cultures are tested in order to detect contamination.

The individual peptide-L-PNA constructs have MW between approx. 4200 and 5000 depending on the composition. Therefore all tests were performed on a molar basis rather than on a weight/volume basis. However, assuming an average MW of the construct of 4500 a concentration of 500 nM equals 2.25 microgram/ml.

Definition of Growth Inhibitory Effect of PNA-Constructs:

The bacterial growth in the wells is described by the lag phase i.e. the period until (before) growth starts, the log phase i.e. the period with maximal growth rate, the steady-state phase followed by the death phase. These parameters are used when evaluating the inhibitory effect of the PNA on the bacterial growth, by comparing growth curves with and without PNA. Total inhibition of bacterial growth is defined as: OD (16 h)=OD (0 h) or no visible growth according to NCCLS Guidelines

In an initial screening the modified PNA molecules are tested in the sensitive 10% medium assay. Positive results are then run in the 100% medium assay in order to verify the inhibitory effect in a more “real” environment (cf. the American guidelines (NCCLS)).

In another aspect of the present invention, the modified PNA molecules can be used to identify preferred targets for the PNA. Based upon the known or partly known genome of the target micro-organisms, e.g. from genome sequencing or cDNA libraries, different PNA sequences can be constructed and linked to an effective anti-infective enhancing Peptide and thereafter tested for its anti-infective activity. It may be advantageous to select PNA sequences shared by as many micro-organisms as possible or shared by a distinct subset of micro-organisms, such as for example Gram-negative or Gram-positive bacteria, or shared by selected distinct micro-organisms or specific for a single micro-organism.

In a further aspect of the present invention, the invention provides a composition for use in inhibiting growth or reproduction of infectious micro-organisms comprising a modified PNA molecule according to the present invention. In one embodiment, the inhibition of the growth of micro-organisms is obtained through treatment with either the modified PNA molecule alone or in combination with antibiotics or other anti-infective agents. In another embodiment, the composition comprises two or more different modified PNA molecules. A second modified PNA molecule can be used to target the same bacteria as the first modified PNA molecule or in order to target different bacteria. In the latter form, specific combinations of target bacteria may be selected to the treatment. Alternatively, the target can be one or more genes, which confer resistance to one or more antibiotics to one or more bacteria. In such a treatment, the composition or the treatment further comprises the use of said antibiotic(s).

In another aspect, the present invention includes within its scope pharmaceutical compositions comprising, as an active ingredient, at least one of the compounds of the general formula (III) or a pharmaceutically acceptable salt thereof together with a pharmaceutically acceptable carrier or diluent.

Pharmaceutical compositions containing a compound of the present invention may be prepared by conventional techniques, e.g. as described in Remington: The Science and Practise of Pharmacy. 19^{th} Ed., 1995. The compositions may appear in conventional forms, for example capsules, tablets, aerosols, solutions, suspensions or topical applications.

Typical compositions include a compound of the invention associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier which may be in the form of a ampoule, capsule, sachet, paper, or other container. When the carrier serves as a diluent, it may be solid, semi-solid, or liquid material which acts as a vehicle, excipient, or medium for the active compound. The active compound can be adsorbed on a granular solid container for example in a sachet. Some examples of suitable carriers are water, salt solutions, alcohols, polyethylene glycols, polyhydroxyethoxylated castor oil, peanut oil, olive oil, gelatine, lactose, terra alba, sucrose, glucose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents, thickeners or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral, nasal, rectal, pulmonary, transdermal or parenteral e.g. depot, subcutaneous, intravenous, intraurethral, intramuscular, intranasal, ophthalmic solution or an ointment, the parenteral or the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a suspension or solution in water or a non-aqueous media, a syrup, emulsion or soft gelatin capsules. Thickeners, flavorings, diluents, emulsifiers, dispersing aids or binders may be added.

For nasal administration, the preparation may contain a compound of formula (III) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

In formulations for treatment or prevention of infectious diseases in mammals the amount of active modified PNA molecules used is determined in accordance with the specific active drug, organism to be treated and carrier of the organism.

Such mammals include also animals, both domestic animals, e.g. household pets, and non-domestic animals such as wildlife.

Usually, dosage forms suitable for oral, nasal, pulmonal or transdermal administration comprise from about 0.01 mg to about 500 mg, preferably from about 0.01 mg to about 100 mg of the compounds of formula (III) admixed with a pharmaceutically acceptable carrier or diluent.

In a still further aspect, the present invention relates to the use of one or more compounds of the general formula (III) or pharmaceutcally acceptable salts thereof for the preparation of a medicament for the treatment and/or prevention of infectious diseases.

In yet another aspect of the present invention, the present invention concerns a method of treating or preventing infectious diseases, which treatment comprises administering to a patient in need of treatment or for prophylactic purposes an effective amount of modified PNA according to the invention. Such a treatment may be in the form of administering a composition in accordance with the present invention. In particular, the treatment may be a combination of traditional antibiotic treatment and treatment with one or more modified PNA molecules targeting genes responsible for resistance to antibiotics.

In yet a further aspect of the present invention, the present invention concerns the use of the modified PNA molecules in disinfecting objects other than living beings, such as surgery tools, hospital inventory, dental tools, slaughterhouse inventory and tool, dairy inventory and tools, barbers and beauticians tools and the like.

Any novel feature or combination of features described herein is considered essential to this invention.

### EXAMPLES

The following examples are merely illustrative of the present invention and should not be considered limiting of the scope of the invention in any way.

The following abbreviations related to reagents are used in the experimental part:

(The monomers and the PNA sequences are stated in bold)

| | |
|---|---|
| A monomer | N-(2-Boc-aminoethyl)-N-(N⁶-(benzyloxycarbon- |
| | yl)adenine-9-yl-acetyl)glycine |
| Boc | Tert butyloxycarbonyl |
| Boc-Lys(2-Cl-Z)-OH | N-α-Boc-N-ε-2-chlorobenzyloxycarbonyl-L- |
| | lysine |
| C monomer | N-(2-Boc-aminoethyl)-N-(N⁴-(benzyloxycarbon- |
| | yl)cytosine-1-yl-acetyl)glycine |
| DCM | Dichloromethane |
| DIEA | N,N-diisopropylethylamine |
| DMF | N,N-dimethylformamide |
| DMSO | Dimethyl sulfoxide |
| G monomer | N-(2-Boc-aminoethyl)-N-(N²-)benzyloxycarbon- |
| | yl)guanine-9-yl-acetyl)glycine |
| HATU | N-[(1-H-benzotriazole-1-yl)(dimethylamine)meth- |
| | ylene]-N-methylmethanaminiumhexafluoro- |
| | phosphate N-oxide |
| HBTU | 2-(1-H-benzotriazole-1-yl)-1,1,3,3-tetramethyl- |
| | uronium hexafluorophosphate |
| J monomer/ | N-(2-Boc-aminoethyl)-N-(N-2-(benzyloxycarbon- |
| nucleobase | yl)isocytosine-5-yl-acetyl)glycine |
| MBHA resin | p-methylbenzhydrylamine resin |
| NMP | N-methyl pyrrolidone |
| T monomer | N-(2-Boc-aminoethyl)-N-(thymine-1-yl-acet- |
| | yl)glycine |
| TFA | Trifluoroacetic acid |
| TFSMA | Trifluoromethanesulphonic acid |
| Tris | 2-amino-2-(hydroxymethyl)-1,3-propanediol |

The composition of mixtures of solvents is indicates on a volume basis, i.e. 30/2/10 (v/v/v).

Preparative HPLC is performed on a DELTA PAK [Waters](C18, 15 μm, 300 Å, 300×7.8 mm, 3 ml/min) A linear gradient from solvent A: 0.1% TFA in water to B: 0.1% TFA in acetonitrile was used. 0-2 min B 10%, 2-30 min 40% B, 30-35 min 100% B, 35-37 min 100% B, 37-38 min 10% B, 37-50 min 10% B.

Mass Spectrometry was performed on MALDI (Matrix Assisted Laser Desorption and Ionisation Time of Flight Mass Spectrometry) as HP MALDI-TOF # G2025A calibrated with peptide nucleic acids of the following weights: Mw₁=1584.5 g/mol, Mw₂=3179.0 g/mol and Mw₃=4605.4 g/mol.

### Example 1

Preparation of Compound No. I: H-KFFKFFKFFK-ado-TTC AAA CAT AGT-NH₂ (SEQ ID No.

The peptide-PNA-chimera H-KFFKFFKFFK-ado-TTC AAA CAT AGT-NH₂ (SEQ ID No.1) was synthesized on 50 mg MBHA resin (loading 100 μmol/g) (novabiochem) in a 5 ml glass reactor with a D-2 glassfilter. Deprotection was done with 2×600 μL TFA/m-cresol 95/5 followed by washing with DCM, DMF, 5% DIEA in DCM and DMF. The coupling mixture was 200 μl 0.26 M solution of monomer (Boc-PNA-T-monomer, Boc-PNA-A-monomer, Boc-PNA-G-monomer, Boc-PNA-C-monomer, Boc-AEEA-OH (ado) (PE Biosystems Inc.)) in NMP mixed with 200 μl 0.5 M DIEA in pyridine and activated for 1 min with 200 μl 0.202 M HATU (PE-biosystems) in NMP. The coupling mixture for the peptide part was 200 μl 0.52 M NMP solution of amino acid (Boc-Phe-OH and Boc-Lys(2-Cl—Z)—OH (novabiochem)) mixed with 200 μl 1 M DIEA in NMP and activated for 1 min with 200 μl 0.45 M HBTU in NMP. After the coupling the resin was washed with DMF, DCM and capped with 2×500 μl NMP/pyridine/acetic anhydride 60/35/5. Washing with DCM, DMF and DCM terminated the synthesis cycle. The oligomer was deprotected and cleaved from the resin using “low-high” TFMSA. The resin was rotated for 1 h with 2 ml of TFA/dimethylsulfid/m-cresol/TFMSA 10/6/2/0.5. The solution was removed and the resin was washed with 1 ml of TFA and added 1.5 ml of TFMSA/TFA/m-cresol 2/8/1. The mixture was rotated for 1.5 h and the filtrated was precipitated in 8 ml diethylether.

The precipitate was washed with 8 ml of diethylether. The crude oligomer was dissolved in water and purified by HPLC. Preparative HPLC was performed on a DELTA PAK [Waters](C18, 15 μm, 300 Å, 300×7.8 mm, 3 ml/min) A linear gradient from solvent A: 0.1% TFA in water to B: 0.1% TFA in acetonitrile was used. 0-2 min B 10%, 2-30 min 40% B, 30-35 min 100% B, 35-37 min 100% B, 37-38 min 10% B, 37-50 min 10% B.

Mw calculated: 4791.9 g/mol; found on MALDI: 4791 g/mol.

### Example 2

Maleimide Activation of PNA

PNA-oligomer ado-TTC AAA CAT AGT-NH₂ (SEQ ID NO: 27) (purified by HPLC) (2 mg, 0.589 μmol, Mw 3396.8) was dissolved and stirred for 15 min in NMP:DMSO 8:2 (2 ml). Succinimidyl 4-(N-maleimidomethyl)cyclohexane-1-carboxylate (SMCC) (PIERCE)(1.1 mg, 3.24 μmol, 5.5 eq.) dissolved in NMP (50 μl) and DIEA (34.7 μl, 198.7 μmol) was added to the solution: The reaction mixture was stirred for further 2.5 h. The product was precipitated in diethylether (10 mL). The precipitate was washed with ether:NMP; 10:1 (3×10 mL) and ether (3×10 mL).

Mw calculated: 3615.8 g/mol; found on MALDI: 3613.5 g/mol.

The product was used without further purification.

### Example 3

Conjugation of Peptide and Maleimide Activated PNA

A solution of peptide CKFFKFFKFFK (SEQ ID NO: 28) (0.5 mg in 200 μl degassed Tris buffer 10 mM, pH 7.6 (329 nM)) was added to a solution of the above activated product (0.2 mg in 200 μl DMF:Water 1:1). The reaction mixture was stirred over night. The target compound was purified by HPLC directly from the crude reaction mixture. Preparative HPLC was performed on a DELTA PAK [Waters](C18, 15 μm, 300 Å, 300×7.8 mm, 3 ml/min) A linear gradient from solvent A: 0.1% TFA in water to B: 0.1% TFA in acetonitrile was used. 0-2 min B 10%, 2-30 min 40% B, 30-35 min 100% B, 35-37 min 100% B, 37-38 min 10% B, 37-50 min 10% B.

Mw calculated: 5133.0 g/mol; found on MALDI: 5133 g/mol.

### Example 4

H-LLKKLAKALKG-ahex-ado-CCATCTAATCCT-NH₂ (SEQ ID NO: 29)

Performed in accordance with example 1, however with the use of 6-aminohexanoic acid (ahex) as linker together with 8-amino-3,6-dioxaoctanoic acid (ado).

### Example 5

Preparation of H-KFFKFFKFF-ado-JTJTJJT-ado-ado-ado-TCCCTCTC-Lys-NH₂ (SEQ ID NO: 30)

Performed in accordance with Example 1, however with the use of PNA oligomer ado -JTJTJJT-ado-ado-ado-TCCCTCTC-Lys-NH₂ (SEQ ID NO: 31) instead of ado-TTC AAA CAT AGT-NH₂ (SEQ ID NO: 27). This PNA is a triplex forming bis-PNA in which C (cytosine) in the “Hoogsteen strand” is exchanged with the J nucleobases (a substitute for protonated C). This substitution assures efficient triplex formation at physiological pH (Egholm, M.; Dueholm, K. L.; Buchardt, O.; Coull, J.; Nielsen, P. E.; Nucleic Acids Research 1995, 23,217-222.

### Example 6

### Preparation of Peptide-PNA-Chimeras

Compound II and III,

(SEQ ID NO: 2)

H-KFFKFFKFFK-C-smcc-ado-TTC AAA CAT AGT-NH₂
and

(SEQ ID NO: 3)

H₂N-KFFKVKFVVKK-C-smcc-ado-TTC AAA CAT AGT-NH₂

were prepared in the same way as described above.

### Example 7

Preparation of KFFKFFKFFK-b.ala-cha-ACTTTGTCGCCA-NH₂ (SEQ ID NO: 32)

The Compound is synthesized on 50 mg MBHA resin (loading 100 μmol/g) in a Teflon reactions vessel. Deprotection is done with 2×600 μL TFA/anisol 95/5 followed by washing with DCM, DMF, 5% DIEA in DCM and DMF. The coupling mixture is 200 μL 0.26 M solution of monomer (Boc-PNA-T-monomer, Boc-PNA-C-monomer, Boc-PNA-A-monomer, Boc-PNA-monomer) in NMP mixed with 200 L 0.5 M DIEA in pyridine and activated for 1 min. with 200 μL 0.202 M HATU in NMP. The coupling mixture for the linker and the peptide part is 200 μL 0.52 M NMP solution of amino acid (Boc-Lys(2-Cl—Z)—OH, Boc-Phe-OH, Boc-cha-OH and Boc-β-Ala-OH) mixed with 200 μL 1 M DIEA in NMP and activated for 1 min. with 200 μL 0.404 M HBTU in NMP. After the coupling the resin is washed with DMF, DCM and capped with 600 μL NMP/Pyridine/acetic anhydride 50/48/2. Washing with DCM, DMF and DCM terminates the synthesis cycle. The oligomer is deprotected and cleaved from the resin using “low-high” TFMSA. The resin is rotated for 1 h with 1 mL of TFA/dimethylsulfid/m-cresol/TFMSA 5.5131110.5. The solution is washed out and then washed with 600 μL of TFA and 1 mL of TFMSA/TFA/m-cresol 2/8/1 is added. The mixture is rotated for 1.5 h and then precipitated out in 8 mL diethylether.

The precipitate is washed with 8 mL of diethylether. The crude compound is dissolved in water and purified by HPLC.

Purity after preparative HPLC 98%, Mw calculated: 4835 g/mol: found on MALDI: 4832 g/mol.

### Example 8

The following Compounds No. IV to VII were prepared as described in Example 7:

Compound No. IV:

(SEQ ID NO: 4)

H-KFFKFFKFFK-b.cypr-g.abu-ACTTTGTCGCCA-NH₂
Compound No. V:

(SEQ ID NO: 5)

H-KFFKFFKFFK-achc-b.Ala-TTCTAACATTTA-NH₂
Compound No. VI:

(SEQ ID NO: 6)

H-KFFKFFKFFK-g.abu-PheGly-TTCTAACATTTA-NH₂
Compound No. VII:

(SEQ ID NO: 7)

H-KFFKFFKFFK-pfPhe-cha-TTCTAACATTTA-NH₂

### Example 9

Bacterial Growth Inhibition with PNA Against the α-Sarcine Loop of Ribosomal RNA.

Compound No. I, II and III.

Growth Assay

The assay was performed as follows:

Dilutions of the test culture E. coli K12 corresponding to, 2×10⁴ and 4×10⁴, cells/ml containing truncated versions of the KFF-motif of the PNA's against α-sarcine loop of ribosomal RNA at a final concentration of 200, 400, 600, 800 and 1000 nM are incubated in 10% Mueller-Hinton broth at 37° C. for 16 hours with constant shaking.

Total inhibition of growth can be seen in cultures with 5×10⁵-5×10⁴ cells/ml and a PNA concentration of at least 200 nM.

### Example 10

Inhibition of Bacterial Growth by PNA-Peptide with Specificity for the Ribosomal α-Sarine Loop

In order to show that the present invention may be used in a treatment of many micro-organisms, a selection of Gram-negative and Gram-positive bacteria were treated under the same assay conditions as used in example 9. The modified PNA molecule used is Compound No. I, II and III.

Gram-Negative Organisms Inhibition of Growth

| | |
|---|---|
| Escherichia coli | + |
| Klebsiella pneumonia | + |
| Pseudomonas aeruginosa | + |
| Salmonella typhimurium | + |
| Gram-positive organisms | |
| Staphylococcus aureus | + |
| Enterococcus faecium | + |
| Micrococcos luteus | + |

All of the bacterial isolates were inhibited. Using the same assay conditions used for testing of E. coli K.12, growth inhibition of different Gram-negative and Gram-positive organisms has been shown.

### Example 11

The oral bioavailability of Compound No. I, II and III was investigated in Sprague-Dawley rats. Rats were dosed intravenously or perorally with approx. 10 mg/kg of the test PNAs. Dose preparations of test substances were made in 5% glucose solution. Plasma samples were collected at intervals 0-4 hours after dosing. Intact test compound was extracted from plasma by a solid-phase extraction procedure and the plasma concentrations determined by HPLC analysis. Areas under the plasma concentration versus time curve (AUC) were calculated by the trapezoidal method and the oral bioavailability calculated as the ratio (AUC[p.o. adm]/AUC[i.v. adm.])×100%, adjusted for actual doses. By this procedure the bioavailability was estimated to be (Compound I): 14%, (Compound II): 15% and (Compound III): 15%.

### Example 12

The subcutaneous bioavailability of Compound No. I and Compound No. III was investigated in Sprague-Dawley rats. Rats were dosed intravenously or subcutaneously with approx. 10 mg/kg of the test PNAs. Formulations for i.v. and s.c. injection were prepared in 5% glucose solution. Plasma samples were collected at intervals 0-4 hours after dosing. Intact test compound was extracted from plasma by a solid-phase extraction procedure and the plasma concentrations determined by HPLC analysis. Areas under the plasma concentration versus time curve (AUC) were calculated by the trapezoidal method and the subcutaneous bioavailability calculated as the ratio (AUC[s.c. adm]/AUC[i.v. adm.])×100%, adjusted for actual doses. By this procedure the subcutaneous bioavailability was estimated to be (Compound I): 105% and (Compound III): 84%.

### Example 13

The oral bioavailability of the Compounds No. IV, V, VI and VII was investigated in NMRI female mice. Mice were dosed intravenously or perorally with approx.

25 mg/kg of test PNAs. Dose preparations of test substance were made in 5% glucose solution. Plasma samples were collected at intervals 0-5 hours after dosing.

Intact test compound was extracted from plasma by a solid-phase extraction procedure and the plasma concentrations determined by HPLC analysis.

Areas under the plasma concentration versus time curve (AUC) were calculated by the trapezoidal method and the oral bioavailability calculated as the ratio (AUC [p.o. adm]/AUC [i.v. adm])×100%, adjusted for actual doses.

By this procedure the bioavailability was estimated to be (Compound IV): 24%, (Compound V): 9%, (Compound VI): 6% and (Compound VII): 54%.

### Example 14

The subcutaneous bioavailability of the Compounds No. IV, V, VI and VII was investigated in NMRI female mice. Mice were dosed intravenously or subcutaneously with approx. 25 mg/kg of test PNAs.

Formulations for i.v. and s.c. injection were prepared in 5% glucose solution. Plasma samples were collected at intervals 0-5 hours after dosing.

Intact test compound was extracted from plasma by a solid-phase extraction procedure and the plasma concentrations determined by HPLC analysis. Areas under the plasma concentration versus time curve (AUC) were calculated by the trapezoidal method and the subcutaneous bioavailability calculated as the ratio (AUC [s.c.adm]/AUC [i.v. adm])×100%, adjusted for actual doses. By this procedure the subcutaneous bioavailability was estimated to be (Compound IV): 78%, (Compound V): 68%, (Compound VI): 62% and (Compound VII): 69%.

### REFERENCES

Davies, J. et al. (1994) Science 264, 375-82.

Nielsen, P. E., Egholm, M., Berg, R. H. and Buchardt, O. Science (1991) 254, 1457-1500.

Egholm, M, Buchardt, O, Christensen, L, Behrens, C, Freier, S. M. Driver, D. A., Berg, R. H., Kim, S. K., Norden, B. and Nielsen, P. E. Nature (1993) 365, 566-568.

Demidov, V., Potaman, V. N., Frank-Kamenetskii, M. D., Egholm, M., Buchardt, O. Sonnichsen, H. S. and Nielsen, P. E. Biochem. Pharmacol. (1994) 48, 1310-1313.

Nielsen, P. E. and Haaima, G. Chemical Society Reviews (1997) 73-78.

Hanvey et al. Science (1992) 258, 1481-5.

Knudsen, H. and Nielsen, P. E. Nucleic Acids Res. (1996) 24, 494-500.

Lewis, L. G. et al. Proc. Natl. Acad. Sci. USA (1996) 93, 3176-81.

Meyer, O. et al. J. Biol. Chem. (1998) 273, 15621-7.

Nyce, J. W. and Metzger, W. J. Nature (1997) 385 721-725.

Pooga, M. et al, Nature Biotechnology (1998) 16, 857-61.

Good, L. & Nielsen, P. E. Proc. Natl. Acad. Sci. USA (1998) 95, 2073-2076.

Good, L. & Nielsen, P. E. Nature Biotechnology (1998) 16, 355-358.

Broome-Smith et al. 1985, Eur J Biochem 147:437-46.

Egholm, M.; Dueholm, K. L.; Buchardt, O.; Coull, J.; Nielsen, P. E.; Nucleic Acids Research 1995, 23,217-222.

1. A pharmaceutical composition comprising a molecule of formula (III):
PEP-L-A (III)
wherein
PEP is any amino acid sequence;
L is a linker or a bond and
A is an antisense agent together with at least one pharmaceutically acceptable carrier, binder, thickener, diluent, buffer, preservative, or surface active agent.

2. A pharmaceutical composition of claim 1 wherein the antisense agent is PNA (Peptide Nucleic Acid) or a modified oligonucleotide selected from the group consisting of Locked Nucleoside Analogues (LNA) or morpholino analogues.

3. A pharmaceutical composition of claim 1 or 2 wherein the antisense agent is PNA and PEP is a cationic peptide or peptide analogue or a functionally similar moiety, the peptide or peptide analogue having the formula (II):
C-(B-A)ₙ-D, (II)
wherein A consists of from 1 to 8 non-charged amino acids and/or amino acid analogs;
B consists of from 1 to 3 positively charged amino acids and/or amino acid analogs;
C consists of from 0 to 4 non-charged amino acids and/or amino acid analogs;
D consists of from 0 to 3 positively charged amino acids and/or amino acid analogs;
n is 1-10;
and the total number of amino acids and/or amino acid analogs is from 3 to 20.

4. A pharmaceutical composition of claim 1 to 3 wherein the linker L is one or more moieties selected from natural occurring or non-natural amino acids or amino acid analogues provided that at least one moiety is selected from non-natural amino acids or amino acid analogues.

5. A pharmaceutical composition of claim 3 or 4 wherein the L is selected from the group consisting of pfPhe (4-fluoro Phenylalanine), pnPhe (4-nitro Phenylalanine), chg (cyclohexyl Glycine), aha (6-amino-hexanoic acid), Gly (Glycine), b.Ala (β-alanine), achc (Cis-5-aminocyclohexanoic acid), cha (β-cyclohexyl alanine), PheGly (Phenylglycine), g.abu (4-aminobutanoic acid), b.cypr (β-cyclopropyl alanine), m.achc (Cis-amino-cyclohexaneacetic acid), 5fPhe (Pentafluoro-Phenylalanine), pmba (4-aminomethyl-benzoic acid), ado ([2-(N-2-amino ethoxy)ethoxy] acetic acid), Nle (Norleucine), Nva (Norvaline), smcc (succinimidyl 4-(N-maleimidomethyl)cyclohexane-1-carboxylate), 4-aminobutyric acid, 4-aminocyclohexylcarboxylic acid, lcsmcc (succinimidyl 4-(N-maleimidomethyl)cyclohexane-1-carboxy-(6-amido-caproate), mbs (succinimidyl m-maleimido-benzoylate), emcs (succinimidyl N-ε-maleimido-caproylate), smph (succinimidyl 6-(β-maleimido-propionamido) hexanoate, amas (succinimidyl N-(α-maleimido acetate), smpb (succinimidyl 4-(p-maleimidophenyl)butyrate) and adc (amino dodecanoic acid) or any combination thereof.

6. A pharmaceutical composition of any of the preceding claims wherein the molecule of formula (III) is selected from the group consisting of:
(SEQ ID NO: 1)
H-KFFKFFKFFK-ado-TTC AAA CAT AGT-NH₂
(SEQ ID NO: 2)
H-KFFKFFKFFK-C-smcc-ado-TTC AAA CAT AGT-NH₂
(SEQ ID NO: 3)
H₂N-KFFKVKFVVKK-C-smcc-ado-TTC AAA CAT AGT-NH₂
(SEQ ID NO: 4)
H-KFFKFFKFFK-b.cypr-g.abu-ACTTTGTCGCCA-NH₂
(SEQ ID NO: 5)
H-KFFKFFKFFK-achc-b.Ala-TTCTAACATTTA-NH₂
(SEQ ID NO: 6)
H-KFFKFFKFFK-g.abu.PheGly-TTCTAACATTTA-NH₂
or
(SEQ ID NO: 7)
H-KFFKFFKFFK-pfPhe-cha-TTCTAACATTTA-NH_{2.}

7. A pharmaceutical composition for therapeutical use comprising a molecule of claim 1 to 6.

8. A pharmaceutical composition according to claim 7 for use in the treatment of infectious diseases.

9. A pharmaceutical composition according to any of the preceding claims in the form of an oral dosage unit.

10. A pharmaceutical composition according to claim 1 to 8 in the form of a parenteral dosage unit.

11. A pharmaceutical composition according to claim 10 in the form of an injectable solution or suspension.

12. A pharmaceutical composition according to any of the preceding claims wherein the molecule is administered as a dose in a range from about 0.01 mg to about 500 mg, preferably from about 0.1 mg to about 100 mg per day.


SEQUENCE LISTING (32 sequences)

SEQ ID NO: 1 - DNA, 12 bp, Artificial Sequence
  Synthetic construct
ttcaaacatagt

SEQ ID NO: 2 - DNA, 12 bp, Artificial Sequence
  Synthetic construct
ttcaaacatagt

SEQ ID NO: 3 - DNA, 12 bp, Artificial Sequence
  Synthetic construct
ttcaaacatagt

SEQ ID NO: 4 - DNA, 12 bp, Artificial Sequence
  Synthetic construct
actttgtcgcca

SEQ ID NO: 5 - DNA, 12 bp, Artificial Sequence
  Synthetic construct
ttctaacattta

SEQ ID NO: 6 - DNA, 12 bp, Artificial Sequence
  Synthetic construct
ttctaacattta

SEQ ID NO: 7 - DNA, 12 bp, Artificial Sequence
  Synthetic construct
ttctaacattta

SEQ ID NO: 8 - protein, 9 aa, Artificial Sequence
  Synthetic construct
KFFKFFKFF

SEQ ID NO: 9 - protein, 8 aa, Artificial Sequence
  Synthetic construct
KFFKFFKF

SEQ ID NO: 10 - protein, 7 aa, Artificial Sequence
  Synthetic construct
KFFKFFK

SEQ ID NO: 11 - protein, 6 aa, Artificial Sequence
  Synthetic construct
KFFKFF

SEQ ID NO: 12 - protein, 5 aa, Artificial Sequence
  Synthetic construct
KFFKF

SEQ ID NO: 13 - protein, 4 aa, Artificial Sequence
  Synthetic construct
KFFK

SEQ ID NO: 14 - protein, 9 aa, Artificial Sequence
  Synthetic construct
FFRFFRFFR

SEQ ID NO: 15 - protein, 9 aa, Artificial Sequence
  Synthetic construct
LLKLLKLLK

SEQ ID NO: 16 - protein, 9 aa, Artificial Sequence
  Synthetic construct
LLRLLRLLR

SEQ ID NO: 17 - protein, 9 aa, Artificial Sequence
  Synthetic construct
LLKKLAKAL

SEQ ID NO: 18 - protein, 11 aa, Artificial Sequence
  Synthetic construct
KRRWPWWPWKK

SEQ ID NO: 19 - protein, 10 aa, Artificial Sequence
  Synthetic construct
KFKVKFVVKK

SEQ ID NO: 20 - protein, 11 aa, Artificial Sequence
  Synthetic construct
LLKLLLKLLLK

SEQ ID NO: 21 - protein, 10 aa, Artificial Sequence
  Synthetic construct
LLKKLAKALK

SEQ ID NO: 22 - protein, 14 aa, Artificial Sequence
  Synthetic construct
RRLFPWWWPFRRVC

SEQ ID NO: 23 - protein, 15 aa, Artificial Sequence
  Synthetic construct
GRRWPWWPWKWPLIC

SEQ ID NO: 24 - protein, 18 aa, Artificial Sequence
  Synthetic construct
LVKKVATTLKKIFSKWKC

SEQ ID NO: 25 - protein, 12 aa, Artificial Sequence
  Synthetic construct
KKFKVKFVVKKC

SEQ ID NO: 26 - protein, 23 aa, Artificial Sequence
  Synthetic construct
GIGKFLHAAKKFAKAFVAEIMNS

SEQ ID NO: 27 - DNA, 12 bp, Artificial Sequence
  Synthetic construct
ttcaaacatagt

SEQ ID NO: 28 - protein, 11 aa, Artificial Sequence
  Synthetic construct
CKFFKFFKFFK

SEQ ID NO: 29 - DNA, 12 bp, Artificial Sequence
  Synthetic construct
ccatctaatcct

SEQ ID NO: 30 - DNA, 15 bp, Artificial Sequence
  Synthetic construct
ntntnnttccctctc

SEQ ID NO: 31 - DNA, 15 bp, Artificial Sequence
  Synthetic construct
ntntnnttccctctc

SEQ ID NO: 32 - DNA, 12 bp, Artificial Sequence
  Synthetic construct
actttgtcgcca